(19) United States
(12) Patent Application Publication
(10) Pub. No.: US 2024/0269207 A1
(43) Pub. Date: Aug. 15, 2024

(54) Cannabis Parthenogenesis Practical Resin And Fiber Cultivation Ptocess

(71) Applicant: Anthony Michael Buckler, White Plains, MD (US)

(72) Inventor: Anthony Michael Buckler, White Plains, MD (US)

(21) Appl. No.: 18/168,821

(22) Filed: Feb. 14, 2023

(51) Int. Cl. A61K 36/185; A01H 5/12; A01H 6/28

(52) U.S. Cl. CPC A61K 36/185; A01H 5/12; A01H 6/28; A61K 2236/30

(57) ABSTRACT

The term “medical cannabis” and “medical marijuana” existed long before any codifying of law. They were around before industry began and the words actually had meaning. Medical cannabis means sensimilla, and sensimilla is a latin word meaning “without seed.” The reason sensimilla is preferred for medicine is simple. Cannabis plants are highly photo-sensitive. This means the plant's entire life cycle must be controlled with light to efficiently and effectively produce medicine. When done properly you will have a vegetative photo-period and a flower photo-period, each being different in duration, intensity, and color of lighting. Working like this allows you to keep a “mother plant” theoretically indefinitely. This allows you to increase plant potency over time and gives you a source for clones. Clones can be developed directly beside the mother until ready to flower. Establishing a mother plant and taking clones allows for a few things. First, you now have a reliable source of female cannabis plants and no seeds are required to be germinated. Second, potency goes up the longer the mother is in the vegetative state. Third, if there are any abnormalities it is easy to spot. Training yourself to cultivate in this manner will make it really easy to identify when “somethings wrong”, and with cannabis something wrong can turn catastrophic quickly. When the female clone has matured and is ready for flowering they are moved away from the mother. They are provided with a different color of light, as well as duration of light and nutrients. When a cannabis plant is flowering in this manner and no male plants are present, and no pollination occurs, it is now in prime condition to provide our medicine, in the form of its resin. In nature, the resin is what the plant uses to entice pollination. If unpollinated, a cannabis plant will freely flow its resin until harvest. If pollinated, it begins to shut down resin flow because it no longer needs to be pollinated. This is when the plant begins to put its energy into seed production and inhibits our medicine. It is particularly this reason why it is imperative for “medical cannabis” to meet the standard of sensimilla.

The normal process by which medical cannabis is cultivated is altered by using LED lighting. Normal cloning attrition rates are significantly higher compared to other methods. However, when using a Cannabis sativa/ruderalis cross-breed known as auto-flowering for its seeds, and an proven effective nutrient schedule designed for resin production, LED lighting may be suitable to produce resin adequately enough to compensate for the loss of flower products for sale. This method would place value on the resin for harvest, the seeds for harvest, and the fiber post process. Given the degenerative effects of LED lighting on cannabis, the morphological changes to the cannabis plant when exposed to LED lighting in as little as two (2) hours, and the “tightening” of the cannabis flower, it appears that the LED exposure is making the fiber of the cannabis plant stronger and more resilient, simply by compacting the plant with light energy. This clearly indicates an increase in the density of the plant's cells and strength of the fiber, with the LED flowering of cannabis being the most beneficial to fiber strength. Currently, hemp fiber is a known commodity and has a market value. When the proper bioengineering of cannabis parthenogenesis is completed, we will have a stronger source of industrial hemp and at a value exceeding the current market. This I believe to be the true benefit of parthenogenetic cannabis and is a development imperative.

### CANNABIS PARTHENOGENESIS PRACTICAL RESIN AND FIBER CULTIVATION PROCESS

The process begins with a sterile growth medium and seeds from Cannabis sativa/ruderalis hybrid known as auto-flowering. Flowering of medicinal parthenogenetic cannabis begins with germinating auto-flowering cannabis seeds. When seedlings are ready they are moved to any red, blue, or red and blue LED lighting system for photoperiods consisting of twelve (12) to eighteen (18) hours a day until the plant's resin indicates the plant has matured, usually ten (10) weeks. After the parthenogenetic cannabis has ripened, the plant is processed for its concentrate within it. The extractants and extraction process can vary, so the products they produce will also vary, and depend on market demand. The seeds are not harvestable with this method. The degenerative nature of LED lighting will stunt seed development if you use a twelve hour photo-period. This process creates cannabis fiber stronger than normal, along with the resin which can be used as medicine.

### CANNABIS PARTHENOGENESIS VEGETATIVE NUTRIENT FORMULA AND PROCESS

During the vegetative cycle nutrients and water are provided every other day. Plants are allowed to drain off any excess not held by the growth medium. Quantities of water consisting of one half to one gallon of water are used once nutrients are mixed and safety thresholds are verified. Starting with one gallon of water the nutrients are added to the water and contained in the following formula; 1.67 ml (2-1-6), 1.67 ml (5-0-1), 1.67 ml (0-6-5), 0.5 ml (0-1-1), 0.33 ml (0.45-0.25-1), 3.33 ml (0-0-1). After a thorough mixing, the solution is tested for PH and adjusted to a PH of 6.5. Depending on the water source the amount of PH adjustment can vary and is not included in the formula.

### CANNABIS PARTHENOGENESIS FLOWER NUTRIENT FORMULA AND PROCESS

All parthenogenetic cannabis flowering consists of a every other day water and nutrient schedule and allowed to drain. All flowering plants receive one gallon of solution. Starting with one gallon of water the nutrients are added in the following formula; 3.75 ml (5-0-1), 2.5 ml (0-6-5), 2.5 ml (0.45-0.25-1), 2.5 ml (0-0-1), 3.75 ml (0-10-0), 2.5 ml (0-18-16), 1 tsp (2-45-28). After thoroughly mixing the solution the PH needs to be checked and adjusted to 6.5 if necessary. This solution can now be given to the plant and will increase the harvestable resin.

1. Process: Cannabis Parthenogenesis Practical Resin And Fiber Cultivation Process

2. Formula: Cannabis Vegetative Process and Solution

3. Formula: Cannabis Flower Process and Solution

4. Parthenogenetic Cannabis Fiber
